(19) United States
(12) Patent Application Publication
(10) Pub. No.: US 2010/0184174 A1
(43) Pub. Date: Jul. 22, 2010

(54) PROCESS FOR THE OXIDATIVE CLEAVAGE OF VINYLAROMATICS USING PEROXIDASES OR LACCASES

(75) Inventors: Wolfgang Kroutil, Graz (AT); Miguel Lara, Graz (AT)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Universitat Graz, Graz (AT)

(21) Appl. No.: 12/668,640

(22) PCT Filed: Jul. 9, 2008
(86) PCT No.: PCT/AT2008/000250

§ 371 Date: Feb. 1, 2010

(30) Foreign Application Priority Data
Jul. 10, 2007 (AT) A 1062/2007
Nov. 23, 2007 (AT) A1902/2007

(51) Int. Cl. C12P 7/24; C12P 7/26

(52) U.S. Cl. 435/148; 435/147

(57) ABSTRACT

The invention relates to a method for the oxidative cleavage of vinyl aromatics of the formula (1) characterized in that (a) compound(s) of the formula (1) is/are oxidized to aldehydes and ketones of the formulas (2) and (3), respectively, in the presence of molecular oxygen using at least one enzyme selected from peroxidases and laccases as a catalyst, according to the following general reaction scheme:

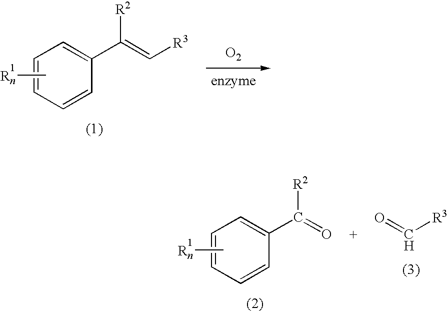

wherein n is an integer of 0 to 5; the R¹ are selected from saturated or unsaturated hydrocarbon groups with 1 to 10 carbon atoms, wherein carbon atoms are optionally substituted by heteroatoms and are optionally further substituted, amino, C₁₋₆ alkylamino and C₁₋₆ dialkylamino groups, halogens, hydroxy and cyano, wherein two of the substituents R¹ may be linked to form a ring; R² and R³ are each independently hydrogen or one of the options for R¹, wherein R² and/or R³ may be linked with R¹ to form a ring, in which case R² and R³ may each represent a chemical bond.

In continuation of this research, the present inventors have now surprisingly found out that, under specific conditions, certain peroxidases and laccases, and not only such of fungal origin, are able to catalyze the oxidative cleavage by oxygen of special ethylenic double bonds to aldehydes and ketones. This result was surprising since oxygen is usually not a substrate (or in the case of laccases at least not a preferred one) for such enzymes and the obtained oxidation products are those usually obtained in ozonolytic reactions. For example, peroxidases can, as the name implies, generally only process peroxide bonds, and halogen peroxidases exclusively result in halogenated, e.g. chlorinated or brominated, oxidation products.

### DISCLOSURE OF THE INVENTION

The present invention provides a method for the oxidative cleavage of ethylenic double bonds conjugated with aromatic rings, i.e. of optionally substituted vinyl aromatics of the following formula (1), by use of at least one metalloprotein as a catalyst, which is characterized in that one or more compound(s) of formula (1) is/are oxidized to aldehydes and ketones of the formulas (2) and (3), respectively, in the presence of molecular oxygen using at least one enzyme selected from peroxidases and laccases as catalyst, according to the following general reaction scheme:

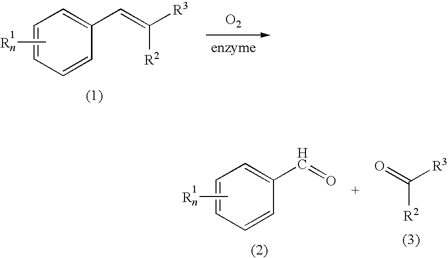

wherein n is an integer of 0 to 5, so that the aromatic ring may be substituted at the ortho, meta and/or para position(s) of the vinyl group with 0 to 5 substituents

### Method for the Oxidative Cleavage of Vinyl Aromatics

The present invention relates to methods for the oxidative cleavage of ethylenic double bonds conjugated with aromatic rings using enzymatic catalysts.

Due to current economic circumstances and an increasing environmental awareness, the demand for mild and selective oxidation methods as well as new ecological and economical chemical methods is higher than ever before. The oxidative cleavage of alkenes into the corresponding aldehydes and ketones is a synthetic method widely used in organic chemistry (i) to introduce oxygen functionalities into molecules, (ii) to split complex molecules into smaller units, and (iii) to remove protective groups. Among the methods currently available for the chemical oxidative cleavage of alkenes, reductive ozonolysis is regarded as the “cleanest”. In practice, however, this method has several disadvantages such as the requirement of using special equipment (ozonator), deep-temperature techniques (usually −78° C.), and the additional need of stoichiometric amounts of reducing agents (e.g. dimethyl sulfide, zinc, hydrogen, phosphines etc.) for the reductive treatment. In addition, particular safety measures have to be taken in order to prevent serious accidents, e.g. by explosions.

In other methods using metal oxides as oxidizing agents, (at least) stoichiometric amounts of salts or peroxides are required. However, these variations show moderate to low chemo-, regio- and stereoselectivities. In many cases, overoxidation of the aldehydes obtained as intermediates to the corresponding acids is a side reaction that is difficult to prevent. For example, the use of OsO₄ and NaIO₄^{[1]}, of OsO₄ and Oxon® (2 KHSO₅+KHSO₄+K₂SO₄)^{[2]}, of RuCl₃ in combination with NaIO₄ or Oxon® ^{[3]}, and of ruthenium nanoparticles with NaIO₄^{[4]} have been described.

Consequently, an oxidation method for alkenes would be desirable, which prevents the above disadvantages and, above all, uses a non-toxic, easily available oxidizing agent such as oxygen.

However, the only known chemical-catalytic method that uses molecular oxygen as an oxidizing agent requires a Co(II) compound as a catalyst, is only moderately selective and is furthermore limited to isoeugenol derivatives^{[5]}.

A possible alternative seems to be biocatalysis. However, enzymatic alkene cleavages have only been described for a few, very specific substrates, using a mixture of lipoxygenases and hydroperoxide lyases^{[6]}.

In addition, the same cleavages have been described as undesired side reactions, i.e. yielding oxidation products in analytical amounts, in peroxidase-catalyzed processes^{[7]-[13]}. Molecular oxygen, of course, has not been used as oxidizing agent in any of these reactions.

Enzymatic alkene cleavage with oxygen by enzymatic catalysis has also been attempted, but only with certain mono- and dioxygenases as enzymes and with yields in analytical amounts^{[14]-[17]}. In addition, oxygenases have very high substrate specifities^{[18]-[29]}, so that only a very limited selection of substrates can be used.

Against this background, the present inventors and their co-workers had already found out in earlier research that certain aryl alkenes can be oxidized to corresponding aldehydes and ketones, using molecular oxygen as the oxidizing agent by adding cells or cell extracts of a certain fungus, Trametes hirsute (white-rot fungus), which catalyze the oxidation^{[30],[31]}. Consequently, this is a biocatalyzed reaction, probably by enzymatic catalysis. However, the enzyme(s) responsible therefor could not be clarified.

In continuation of this research, the present inventors have now surprisingly found out that, under specific conditions, certain peroxidases and laccases, and not only such of fungal origin, are able to catalyze the oxidative cleavage by oxygen of special ethylenic double bonds to aldehydes and ketones. This result was surprising since oxygen is usually not a substrate (or in the case of laccases at least not a preferred one) for such enzymes and the obtained oxidation products are those usually obtained in ozonolytic reactions. For example, peroxidases can, as the name implies, generally only process peroxide bonds, and halogen peroxidases exclusively result in halogenated, e.g. chlorinated or brominated, oxidation products.

### DISCLOSURE OF THE INVENTION

The present invention provides a method for the oxidative cleavage of ethylenic double bonds conjugated with aromatic rings, i.e. of optionally substituted vinyl aromatics of the following formula (1), which is characterized in that one or more compound(s) of formula (1) is/are oxidized to aldehydes and ketones of the formulas (2) and (3), respectively, in the presence of molecular oxygen using at least one enzyme selected from peroxidases and laccases as a catalyst, according to the following general reaction scheme:

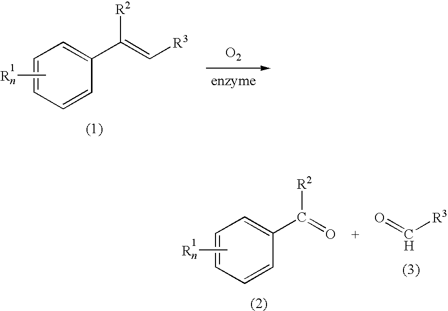

wherein n is an integer of 0 to 5, so that the aromatic ring may be substituted at the ortho, meta and/or para position(s) of the vinyl group with 0 to 5 substituents R¹ which may be identical or different and are selected from:
a) saturated or unsaturated hydrocarbon groups having 1 to 10 carbon atoms, wherein one or more carbon atoms are optionally substituted by a heteroatom selected from oxygen, nitrogen and sulfur, and which are optionally further substituted with one or more substituents selected from C₁₋₆ alkyl groups, C₁₋₆ alkylene groups, C₁₋₆ alkoxy groups, amino, C₁₋₆ alkylamino and C₁₋₆ dialkylamino groups, halogens, hydroxy, oxo and cyano,
b) amino, C₁₋₆ alkylamino and C₁₋₆ dialkylamino groups, and
c) halogens, hydroxy and cyano,
   wherein any two of the substituents R¹ may be linked to form an alicyclic or aromatic ring, and
   wherein the substituents R² and R³ are each independently hydrogen or one of the options described in a), b) and c),
   wherein R² and/or R³ may be linked to a substituent R¹ to form an alicyclic ring, in which case R² and R³ may each represent a chemical bond between the carbon atom of the vinyl group to which they are bound and the substituent R¹.

Thus, an oxidation method for the above compounds is provided, by means of which the aim defined above can be achieved. This means that aryl alkenes can be oxidized to the desired aldehydes and ketones such as vanillin by use of oxygen, an omnipresent, harmless oxidizing agent, and specific natural enzymes which are easily and economically available through biological or biotechnological means. Thus, neither expensive or toxic (heavy metal) catalysts nor complicated and expensive equipment (ozonator, deep-temperature cooling systems) are required, and no waste products with complicated disposal requirements are obtained.

In preferred embodiments, the at least one enzyme is selected from fungal peroxidases and laccases, halogen peroxidases, lignin peroxidases, horseradish peroxidase and bovine milk peroxidase, more preferably from fungal peroxidases from Coprinus cinereus (inky cap fungus), from laccases from Coriolus versicolor (turkey tail), Agaricus bisporus (cultivated mushroom) and Candida rugosa (a yeast species), laccase from Rhus vernicifera (Japanese lacquer tree), chloroperoxidase from Caldariomyces fumago (a filamentous fungus) and bromoperoxidases, e.g. from Streptomyces aureofaciens (a bacterium) or Corallina officinalis (coral seaweed), most preferably from horseradish peroxidase, from peroxidases from Coprinus cinereus as well as laccases from Coriolus versicolor and Agaricus bisporus. Generally, preferred peroxidases are those of the EC class 1.11.1.x. The above enzymes allow—under respectively optimized conditions as explained below—very good results.

Preferably, the method is carried out in a buffer in order to be able to maintain stable reaction conditions, especially a stable pH, during oxidation. Preferably, the reaction is conducted in Bis-Tris buffer, acetate buffer, formate buffer or phosphate buffer. The pH value of the reaction mixture is preferably adjusted to 2 to 7, more preferably to 2 to 4, as the enzymes show their respective maximum activities in these ranges.

In a further preferred embodiment, the inventive method is carried out at an O₂ overpressure to increase the yields. This, however, is not simply the result of a shift of the reaction equilibrium, as can be seen by the fact that some enzymes, having reached an activity maximum, showed lower yields again at higher pressures. Preferably, oxidation is carried out at an O₂ overpressure of 1 to 6 bar, preferably 2 to 3 bar. Higher values do not or hardly result in additional improvements, often even in lower conversions, and would considerably increase equipment requirements. In the above pressure ranges, for example, a conventional Parr apparatus can be used without difficulty.

In further preferred embodiments, the method is carried out under the action of light, i.e. with irradiation, as this allows a multiple increase of the yields, especially when laccases are used as enzymes.

In additional preferred embodiments, the method is carried out in the presence of an organic solvent or solvent mixture, which is preferably selected from C₁₋₄ alkanols, dimethyl sulfoxide, toluene, acetone, dioxane, tetrahydrofuran, dimethyl formamide and mixtures thereof, and its content is preferably 1 to 20% by volume, more preferably 5 to 15% by volume, of the reaction mixture.

In a further aspect, the invention thus concerns the use of fungal peroxidases and laccases, fungal halogen peroxidases, bacterial halogen peroxidases, lignin peroxidases, horseradish peroxidase or bovine milk peroxidase for the catalysis of the oxidative cleavage of ethylenic double bonds conjugated with the aromatic ring of optionally substituted vinyl aromatics by molecular oxygen, wherein the same enzymes are preferred as described for the method according to the first aspect of the invention.

### SHORT DESCRIPTION OF THE DRAWINGS

FIGS. 1 to 3 show the conversion changes in the inventive method with the addition of various organic solvents.

The invention will now be described in more detail by means of specific examples, which are provided for illustration purposes only and not for limitation.

### EXAMPLES

Once the reactivity of various enzymes as catalysts of the oxidation of aryl alkenes by molecular oxygen had been determined in preliminary experiments, the pH optimum of the individual enzymes for such reactions was established in a model reaction using trans-anethole as the vinyl aromatic of formula (1). For adjusting the pH to values from 2 to 7, known buffer systems were used.

### Examples 1 to 16

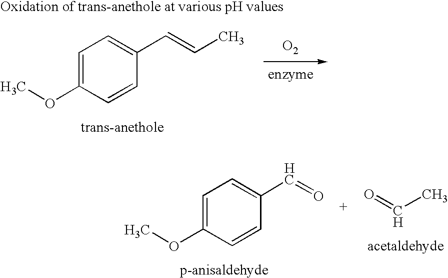

The respective enzymes (3 mg each of the preparations, which were all solid) were placed into the wells of a “Riplate LV” 5 ml Deep Well Plate (HJ-Bioanalytik GmbH). Subsequently, 900 μl of the respective buffers and 6 μl (0.04 mmol) of trans-anethole were added. The plates were then placed into an O₂ pressure reactor in an upright position. The reactor was purged with pure molecular oxygen, and the pressure was adjusted to 2 bar oxygen. After 24 h at 170 rpm and 25° C., the reaction mixtures were transferred into 2 ml test tubes, and the wells were washed with EtOAc (600 μl). These 600 μl were added to the respective test tubes in order to also carry out a first extraction of the aqueous reaction mixtures therewith. After a second extraction with pure EtOAc (600 μl), the combined organic layers were dried over Na₂SO₄ and analyzed for the conversion to p-anisaldehyde (4-methoxy benzaldehyde) by GC.

The buffers for adjusting the pH values were the following:

pH 2 —trimethylammonium formate/formic acid, 20 mM
pH 3 —trimethylammonium formate/formic acid, 20 mM
pH 4 —sodium acetate/acetic acid, 50 mM
pH 5 —sodium acetate/acetic acid, 50 mM
pH 6 —Bis-Tris buffer, 50 mM
pH 7 —Bis-Tris buffer, 50 mM

The enzymes used in the respective examples and the conversions of trans-anethole to p-anisaldehyde obtained at various pH values are shown in the following Table 1.

TABLE 1

| | | Conversion to p-anisaldehyde (%) | | | | | |
|---|---|---|---|---|---|---|---|
| Ex. | Enzyme | pH 2 | pH 3 | pH 4 | pH 5 | pH 6 | pH 7 |
| 1 | peroxidase from Coprinus cinereus, batch 1 | 62 | 68 | 73 | 6 | 3 | 6 |
| 2 | peroxidase from Coprinus cinereus, batch 2 | 7 | 7 | 73 | 5 | 4 | 7 |
| 3 | horseradish peroxidase, batch 1 | 63 | 88 | 65 | 8 | 8 | 14 |
| 4 | horseradish peroxidase, batch 2 | 76 | 57 | 64 | 10 | 13 | 6 |
| 5 | horseradish peroxidase, batch 3 | 88 | 55 | 53 | 3 | 4 | 5 |
| 6 | horseradish peroxidase, batch 4 | 95 | 93 | 73 | 7 | 8 | 5 |
| 7 | horseradish peroxidase, batch 5 | 83 | 64 | 79 | 6 | 7 | 11 |
| 8 | horseradish peroxidase, batch 6 | 86 | 82 | 30 | 16 | 11 | 11 |
| 9 | lignin peroxidase | 10 | 4 | 67 | 25 | 9 | 4 |
| 10 | bovine milk peroxidase | ND | 3 | 4 | 4 | 2 | ND |
| 11 | chloroperoxidase from Caldariomyces | ND | 3 | 4 | 3 | 2 | ND |
| | fumago | | | | | | |
| 12 | bromoperoxidase from Corallina officinalis | 3 | 4 | 5 | 3 | 3 | 3 |
| 13 | laccase from Rhus vernicifera | ND | ND | 2 | 3 | 2 | ND |
| 14 | laccase from Coriolus versicolor | ND | ND | 2 | 4 | 3 | ND |
| 15 | laccase from Candida Rugosa | ND | ND | 3 | 3 | 2 | ND |
| 16 | laccase from Agaricus bisporus | 8 | 6 | 4 | 10 | 10 | 6 |

ND.: not determined

This table clearly shows the surprising catalytic effect of the peroxidases, halogen peroxidases and laccases tested in the above oxidation reaction. Compared to halogen peroxidases and laccases, peroxidases are more reactive, but the conversions of some other enzymes, especially of Agaricus bisporus laccase, are absolutely sufficient for a preparative implementation of the inventive method without the necessity of carrying out one of the optimizations described further below.

Next, the effect of oxygen pressure on the performance of the enzymes as biocatalysts was tested.

### Examples 17-31

### Oxidation of Trans-Anethole at Various Oxygen Pressures

Essentially, the reactions and GC measurements were carried out as in Examples 1 to 16, with the exception that the pressure for each enzyme tested was varied between 2 and 6 bar. Due to the extensive equipment requirements, higher pressures were not examined. The results of the tests are shown in the following Table 2.

TABLE 2

| | | Conversion to p-anisaldehyde (%) | | | | |
|---|---|---|---|---|---|---|
| Ex. | Enzyme | 1 bar | 2 bar | 3 bar | 4 bar | 6 bar |
| 17 | peroxidase from Coprinus cinereus, batch 1 | 69 | 63 | 75 | 68 | 76 |
| 18 | peroxidase from Coprinus cinereus, batch 2 | 33 | 61 | 48 | 34 | 45 |
| 19 | peroxidase from Coprinus cinereus, batch 3 *) | 12 | 8 | 47 | 53 | 15 |
| 20 | horseradish peroxidase, batch 1 | 65 | 70 | 76 | 68 | 57 |
| 21 | horseradish peroxidase, batch 2 | 63 | 63 | 76 | 70 | 75 |
| 22 | horseradish peroxidase, batch 3 | 4 | 23 | 22 | 4 | 17 |
| 23 | horseradish peroxidase, batch 4 | 65 | 83 | 73 | 47 | 75 |
| 24 | horseradish peroxidase, batch 5 | 5 | 47 | 14 | 3 | 4 |
| 25 | horseradish peroxidase, batch 6 | 61 | 68 | 77 | 68 | 69 |
| 26 | lignin peroxidase | 6 | 67 | 23 | 4 | 4 |
| 27 | laccase from Rhus vernicifera | 3 | 3 | 9 | 3 | 3 |
| 28 | laccase from Agaricus bisporus | 3 | 3 | 12 | 3 | 4 |
| 29 | laccase from Coriolus versicolor | 4 | 8 | 5 | 3 | 8 |
| 30 | laccase from Candida Rugosa | 7 | 3 | 7 | 6 | 9 |
| 31 | DeniLite II Base laccase | 3 | 3 | 5 | 3 | 3 |

*) Liquid preparation, of which 20 μl (instead of 3 mg) were used.

It is noticeable that no general preference for higher or lower pressures can be identified. The expectation that higher oxygen pressures would shift the reaction equilibrium towards the products side was not fulfilled. Rather, it seems that each enzyme does not only have an optimum pH range, but also an optimum pressure range.

In further experiments, the various enzymes were tested with different substrates, e.g. aryl alkenes of formula (1), under otherwise substantially equal conditions in order to be able to deduce substrate specifities. The only exception was that the reactions were carried out at the respective enzymes' pH optima, as previously determined.

### Examples 32 to 36

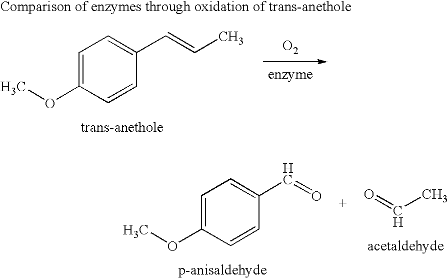

The reactions, work-ups and GC measurements were conducted as described for Examples 1 to 16 (2 bar oxygen) and using a buffer corresponding to the respective pH optimum. The enzymes, buffers, pH values used and the conversions of trans-anethole to p-anisaldehyde are shown in the following Table 3.

TABLE 3

| | | | | Conversion to |
|---|---|---|---|---|
| Ex. | Enzyme | Buffer | pH | anisaldehyde (%) |
| 32 | peroxidase from Coprinus cinereus, batch 1 | Me₃N/HCOOH | 2 | 61 |
| 33 | horseradish peroxidase, batch 1 | Me₃N/HCOOH | 2 | 83 |
| 34 | lignin peroxidase | AcONa/AcOH | 4 | 67 |
| 35 | laccase from Agaricus bisporus | AcONa/AcOH | 5 | 10 |
| 36 | laccase from Coriolus versicolor | AcONa/AcOH | 5 | 8 |

Again, it was clearly shown that trans-anethole can be oxidized to p-anisaldehyde by enzymatic catalysis with sometimes very good yields, and that peroxidases are clearly superior to laccases, even though the latter can also be used for preparative purposes.

### Examples 37 to 40

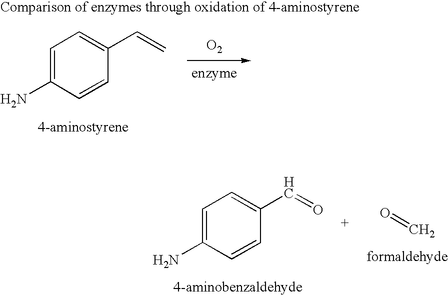

By analogy with Examples 32 to 36, 4-aminostyrene instead of trans-anethole was oxidized to 4-aminobenzaldehyde, using different enzymes and different buffers. In addition, during work-up, the pH of the aqueous phase was adjusted to 10 in order to prevent salification of the amino groups. The results of the experiments and the GC measurements are shown in Table 4.

TABLE 4

| | | | | Conversion to |
|---|---|---|---|---|
| | | | | aminobenzaldehyde |
| Ex. | Enzyme | Buffer | pH | (%) |
| 37 | horseradish peroxidase, | Me₃N/HCOOH | 2 | 3 |
| | batch 1 | | | |
| 38 | lignin peroxidase | AcONa/AcOH | 4 | 2 |
| 39 | laccase from | AcONa/AcOH | 5 | 3 |
| | Agaricus bisporus | | | |
| 40 | laccase from | AcONa/AcOH | 5 | 19 |
| | Coriolus versicolor | | | |

It has been shown that, under the experimental conditions, among the four enzymes tested only laccase from Coriolus versicolor could catalyze the oxidation of 4-aminostyrene with good conversion results. This fact and the fact that the enzyme developed more than twice its efficiency compared to the case of trans-anethole clearly prove that the enzymes show substrate specifity.

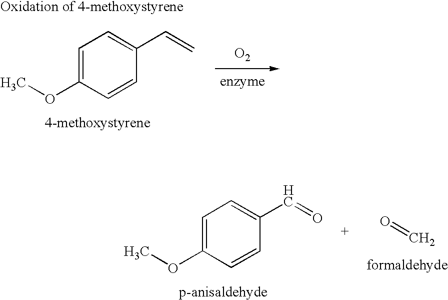

### Example 41

By analogy with Example 32, 4-methoxystyrene instead of trans-anethole was oxidized with the peroxidase from Coprinus cinereus, batch 1, to p-anisaldehyde. The results of the two experiments are shown in Table 5.

TABLE 5

| | | | | Conversion to |
|---|---|---|---|---|
| Ex. | Enzyme | Buffer | pH | anisaldehyde (%) |
| 32 | peroxidase from | Me₃N/HCOOH | 2 | 61 |
| | Coprinus cinereus, batch 1 | | | |
| 41 | peroxidase from | Me₃N/HCOOH | 2 | 3 |
| | Coprinus cinereus, batch 1 | | | |

This example again gives evidence for the substrate specifity of the enzymes. Obviously, the peroxidase tested is able to oxidize very well the double bond of trans-anethole substituted on both sides, however, hardly oxidizes that of methoxystyrene, being unsubstituted on one side. Thus, the substituents of the vinyl group have a substantial effect on the catalytic reaction.

### Examples 42 and 43

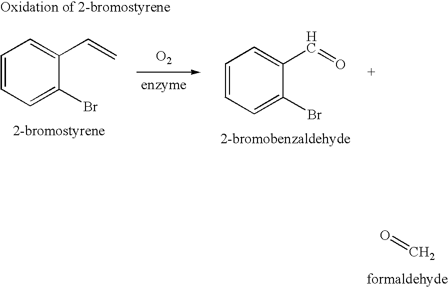

By analogy with Examples 32 and 33, 2-bromostyrene instead of trans-anethole was oxidized with the peroxidase from Coprinus cinereus, batch 1, and the horseradish peroxidase, batch 1, in this case to 2-bromobenzaldehyde. The results of the four experiments are shown in Table 6.

TABLE 6

| | | | | Conversion to |
|---|---|---|---|---|
| Ex. | Enzyme | Buffer | pH | product (%) |
| | | | | p-anisaldehyde |
| 32 | peroxidase from Coprinus cinereus, batch 1 | Me₃N/HCOOH | 2 | 61 |
| 33 | horseradish peroxidase, batch 1 | Me₃N/HCOOH | 2 | 83 |
| | | | | 2-bromobenzaldehyde |
| 42 | peroxidase from Coprinus cinereus, batch 1 | Me₃N/HCOOH | 2 | 4 |
| 43 | horseradish peroxidase, batch 1 | Me₃N/HCOOH | 2 | 3 |

This result again proves that substituents at the aromatic ring also have a substantial effect on the catalytic reaction.

### Example 44

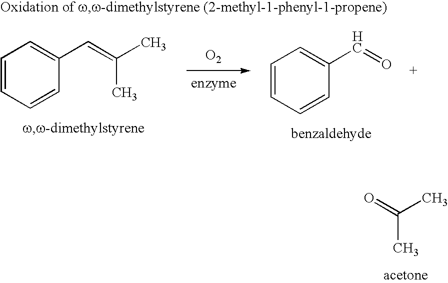

By analogy with Example 33, ω,ω-dimethylstyrene instead of trans-anethole was oxidized with horseradish peroxidase, batch 1, in this case to benzaldehyde. The results of the two experiments and those of Example 43 are shown in Table 7 for comparative purposes.

TABLE 7

| | | | | Conversion to |
|---|---|---|---|---|
| Ex. | Enzyme | Buffer | pH | product (%) |
| | | | | p-anisaldehyde |
| 33 | horseradish peroxidase, batch 1 | Me₃N/HCOOH | 2 | 83 |
| | | | | 2-bromobenzaldehyde |
| 43 | horseradish peroxidase, batch 1 | Me₃N/HCOOH | 2 | 3 |
| | | | | benzaldehyde |
| 44 | horseradish peroxidase, batch 1 | Me₃N/HCOOH | 2 | 5 |

Again, evidence for the substrate specifity was provided. The presence of two methyl groups at the ω-carbon of styrene instead of only one and the lack of substituents at the aromatic ring have a substantial effect on the catalysis.

### Examples 45 to 47

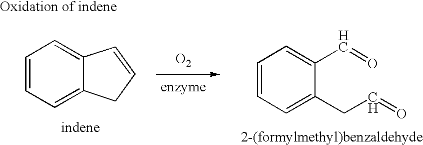

By analogy with Examples 32, 33 and 36, indene instead of trans-anethole was oxidized with the peroxidase from Coprinus cinereus, batch 1, horseradish peroxidase, batch 1, and the laccase from Coriolus versicolor, in this case to 2-(formylmethyl)benzaldehyde. The results of the six experiments are shown in Table 8.

TABLE 8

| | | | | Conversion to |
|---|---|---|---|---|
| Ex. | Enzyme | Buffer | pH | product (%) |
| | | | | p-anisaldehyde |
| 32 | peroxidase from Coprinus cinereus, batch 1 | Me₃N/HCOOH | 2 | 61 |
| 33 | horseradish peroxidase, batch 1 | Me₃N/HCOOH | 2 | 83 |
| 36 | laccase from Coriolus versicolor | AcONa/AcOH | 5 | 8 |
| | | | | 2-(formylmethyl)- |
| | | | | benzaldehyde |
| 45 | peroxidase from Coprinus cinereus, batch 1 | Me₃N/HCOOH | 2 | 4 |
| 46 | horseradish peroxidase, batch 1 | Me₃N/HCOOH | 2 | 12 |
| 47 | laccase from Coriolus versicolor | AcONa/AcOH | 5 | 8 |

While the two peroxidases had clearly lower catalytic activity when indene was used as a substrate, the (less active) laccase showed hardly any difference compared to trans-anethole. However, it is proven that ethylenic double bonds contained in cyclic structures may also be oxidized according to the inventive method.

### Comparative Examples 1 and 2

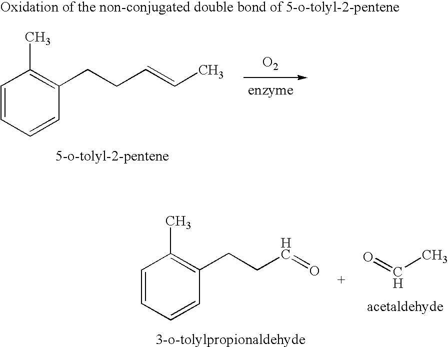

By analogy with Examples 32 and 33, 5-o-tolyl-2-pentene instead of trans-anethole was oxidized with the peroxidase from Coprinus cinereus, batch 1, and horseradish peroxidase, batch 1, in this case to 3-o-tolylpropionaldehyde. The results of the two experiments are shown in Table 9.

TABLE 9

| | | | | Conversion to 3-o- |
|---|---|---|---|---|
| Comp. | | | | tolylpropionaldehyde |
| Ex. | Enzyme | Buffer | pH | (%) |
| 1 | peroxidase from Coprinus cinereus, batch 1 | Me₃N/HCOOH | 2 | <1 |
| 2 | horseradish peroxidase, batch 1 | Me₃N/HCOOH | 2 | <1 |

These results show that arylalkenes with non-conjugated double bonds cannot be oxidized with the inventive method. The oxidation products are only detectable in analytic amounts (≦1%).

### Examples 48 to 51

### Oxidation of Trans-Anethole Under Various Light Conditions

The reactions and GC measurements were conducted in duplicate for each enzyme tested, essentially as described in Examples 32 to 36 in the buffers indicated therein for the respective enzymes. However, in a first experimental series, a lamp (PAR 38 EC Spot, Osram Concentra, 120 W, 230 V, 448) was arranged at a distance of 50 cm above the reactor in order to illuminate the reaction mixtures during oxidation, while in a second experimental series the reactor was covered with an aluminum foil for darkening, which foil was perforated to allow oxygen exchange with the environment. The results of the best four of all enzymes tested are shown in the following Table 10.

TABLE 10

| | | Conversion to | |
|---|---|---|---|
| | | anisaldehyde (%) | |
| Ex. | Enzyme | illuminated | dark |
| 48 | peroxidase from Coprinus cinereus, batch 1 | 85 | 43 |
| 49 | horseradish peroxidase, batch 1 | 86 | 55 |
| 50 | laccase from Agaricus bisporus | 32 | 4 |
| 51 | laccase from Coriolus versicolor | 64 | 7 |

The results show a clear increase of the catalytic activity of all four enzymes under the action of light. This effect is particularly well pronounced with the two laccases, since their effectiveness was increased to the eight- or ninefold level. Thus, generally less active laccases can also result in very good conversions for preparative purposes.

### Examples 52 to 85

### Oxidation of Trans-Anethole in the Presence of Various Organic Solvents

By analogy with the method described for Examples 1 to 16, trans-anethole was oxidized by means of peroxidase from Coprinus cinereus, batch 1, or horseradish peroxidase, batch 1, as a catalyst. The conversions thus obtained to p-anisaldehyde of 44% and 58%, respectively, served as blanks for subsequent repetitions of the methods, wherein, however, in each case 17 μl of an organic solvent were added to the 900 μl of the aqueous trimethylammonium formate/formic acid buffer (20 mM).

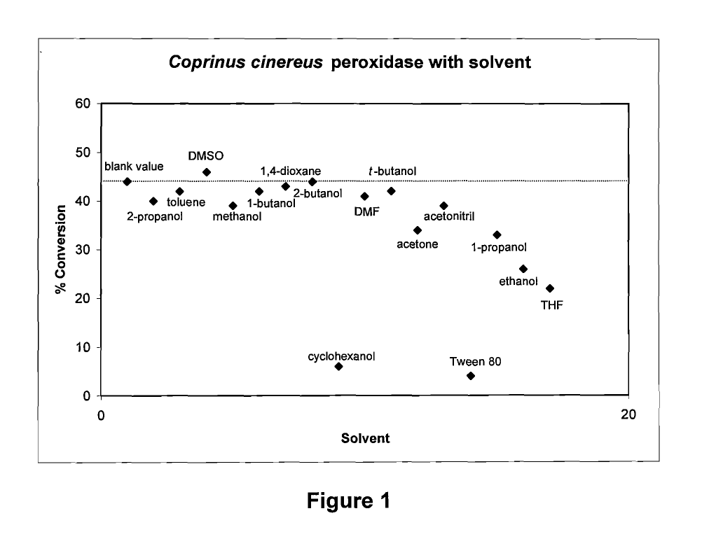

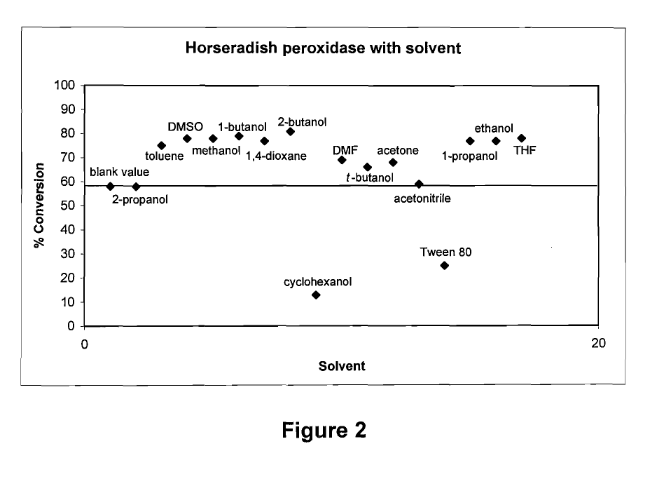

The results are shown in the following Tables 11 and 12 and shown graphically in FIGS. 1 and 2, wherein the horizontal lines show the values of experiments without solvent (“blank”).

TABLE 11

| | | | | Conversion to |
|---|---|---|---|---|
| Ex. | Enzyme | Solvent | pH | p-anisaldehyde (%) |
| 52 | peroxidase from Coprinus cinereus, batch 1 | none | 2 | 44 |
| 53 | peroxidase from Coprinus cinereus, batch 1 | 2-propanol | 2 | 40 |
| 54 | peroxidase from Coprinus cinereus, batch 1 | toluene | 2 | 42 |
| 55 | peroxidase from Coprinus cinereus, batch 1 | DMSO | 2 | 46 |
| 56 | peroxidase from Coprinus cinereus, batch 1 | methanol | 2 | 39 |
| 57 | peroxidase from Coprinus cinereus, batch 1 | 1-butanol | 2 | 42 |
| 58 | peroxidase from Coprinus cinereus, batch 1 | 1,4-dioxane | 2 | 43 |
| 59 | peroxidase from Coprinus cinereus, batch 1 | 2-butanol | 2 | 44 |
| 60 | peroxidase from Coprinus cinereus, batch 1 | cyclohexanol | 2 | 6 |
| 61 | peroxidase from Coprinus cinereus, batch 1 | DMF | 2 | 41 |
| 62 | peroxidase from Coprinus cinereus, batch 1 | t-butanol | 2 | 42 |
| 63 | peroxidase from Coprinus cinereus, batch 1 | acetone | 2 | 34 |
| 64 | peroxidase from Coprinus cinereus, batch 1 | acetonitrile | 2 | 39 |
| 65 | peroxidase from Coprinus cinereus, batch 1 | Tween 80 | 2 | 4 |
| 66 | peroxidase from Coprinus cinereus, batch 1 | 1-propanol | 2 | 33 |
| 67 | peroxidase from Coprinus cinereus, batch 1 | ethanol | 2 | 26 |
| 68 | peroxidase from Coprinus cinereus, batch 1 | THF | 2 | 22 |

TABLE 12

| | | | | Conversion to |
|---|---|---|---|---|
| | | | | p-anisaldehyde |
| Ex. | Enzyme | Solvent | pH | (%) |
| 69 | horseradish peroxidase, batch 1 | none | 2 | 58 |
| 70 | horseradish peroxidase, batch 1 | 2-propanol | 2 | 58 |
| 71 | horseradish peroxidase, batch 1 | toluene | 2 | 75 |
| 72 | horseradish peroxidase, batch 1 | DMSO | 2 | 78 |
| 73 | horseradish peroxidase, batch 1 | methanol | 2 | 78 |
| 74 | horseradish peroxidase, batch 1 | 1-butanol | 2 | 79 |
| 75 | horseradish peroxidase, batch 1 | 1,4-dioxane | 2 | 77 |
| 76 | horseradish peroxidase, batch 1 | 2-butanol | 2 | 81 |
| 77 | horseradish peroxidase, batch 1 | cyclohexanol | 2 | 13 |
| 78 | horseradish peroxidase, batch 1 | DMF | 2 | 69 |
| 79 | horseradish peroxidase, batch 1 | t-butanol | 2 | 66 |
| 80 | horseradish peroxidase, batch 1 | acetone | 2 | 68 |
| 81 | horseradish peroxidase, batch 1 | acetonitrile | 2 | 59 |
| 82 | horseradish peroxidase, batch 1 | Tween 80 | 2 | 25 |
| 83 | horseradish peroxidase, batch 1 | 1-propanol | 2 | 77 |
| 84 | horseradish peroxidase, batch 1 | ethanol | 2 | 77 |
| 85 | horseradish peroxidase, batch 1 | THF | 2 | 78 |

DMSO: dimethyl sulfoxide;
DMF: dimethyl formamide;
Tween 80: polyoxyethylene(20) sorbitan monooleate;
THF: tetrahydrofuran

All results prove that the presence of an organic solvent is basically possible without completely inhibiting the oxidation reaction. As may be seen from Table 11 and FIG. 1, peroxidase from Coprinus cinereus is more sensitive to the addition of a solvent because the conversions are all lower due to the solvents—with the exception of DMSO.

In contrast, the addition of a solvent mostly leads to a conversion increase when horseradish peroxidase is used as the catalyst. Only in the cases of cyclohexanol and Tween 80, there is a decrease, and 2-propanol provides the same result as the blank.

Without wishing to be bound by any special theory, it is assumed that here the solvent serves as solubilizer for the compound of formula (1) to be oxidized, even though only 1.8% by volume of a solvent were added to the aqueous buffer. In order to investigate if higher amounts of a solvent would also have a positive effect on the conversion using horseradish peroxidase, a further test series was conducted with increasing amounts of DMSO, the only solvent that had shown a positive effect with peroxidase from Coprinus cinereus.

### Examples 86 to 96

### Oxidation of Trans-Anethole in the Presence of Increasing Amounts of DMSO

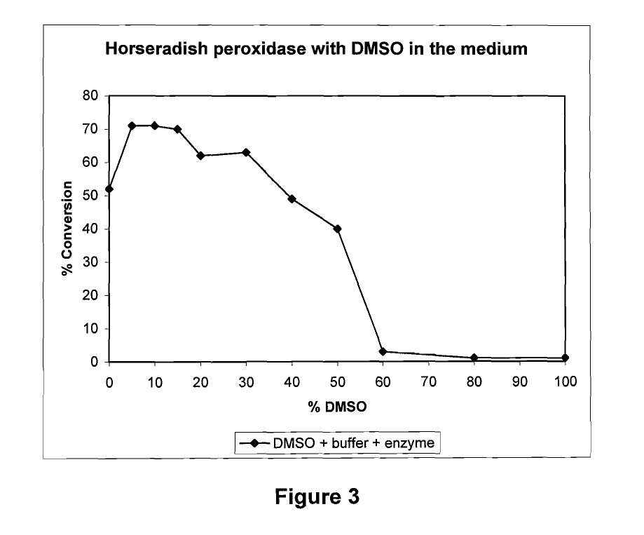

By analogy with the above Examples 69 to 85, trans-anethole was oxidized with horseradish peroxidase as a catalyst, wherein the 900 μl of aqueous buffer were replaced by increasing percentages of DMSO. In Table 13, the conversions achieved at the respective contents of DMSO are shown. In FIG. 3, the data are also shown graphically.

TABLE 13

| | | Content of DMSO | | Conversion to |
|---|---|---|---|---|
| Ex. | Enzyme | in the medium (%) | pH | p-anisaldehyde (%) |
| 86 | horseradish peroxidase, batch 1 | 0 | 2 | 52 |
| 87 | horseradish peroxidase, batch 1 | 5 | 2 | 71 |
| 88 | horseradish peroxidase, batch 1 | 10 | 2 | 71 |
| 89 | horseradish peroxidase, batch 1 | 15 | 2 | 70 |
| 90 | horseradish peroxidase, batch 1 | 20 | 2 | 62 |
| 91 | horseradish peroxidase, batch 1 | 30 | 2 | 63 |
| 92 | horseradish peroxidase, batch 1 | 40 | 2 | 49 |
| 93 | horseradish peroxidase, batch 1 | 50 | 2 | 40 |
| 94 | horseradish peroxidase, batch 1 | 60 | 2 | 8 |
| 95 | horseradish peroxidase, batch 1 | 80 | 2 | 8 |
| 96 | horseradish peroxidase, batch 1 | 100 | 2 | 8 |

It may be seen that, at a content of 40% of DMSO in the medium, horseradish peroxidase shows approximately the same activity as without organic solvent. At higher concentrations the activity decreases quickly, and from 60% of DMSO onward substantially no enzymatic effect is detectable. With 20 to 30% of DMSO in the medium, the conversion was increased by approximately 20%. The best results, i.e. a conversion increase of approximately 40%, was achieved with 5 to 15% of the solvent.

To verify if this effect is also detectable in other reactions, the following test series for producing vanillin (4-hydroxy-3-methoxybenzaldehyde), one of the possible valuable reaction products of the inventive method, was conducted.

### Examples 97 to 104

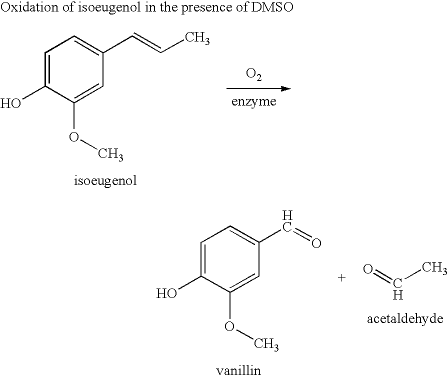

By analogy with Examples 52 to 85, isoeugenol (2-methoxy-4-propene-1-ylphenol) instead of trans-anethole was oxidized with peroxidase from Coprinus cinereus or horseradish peroxidase as catalyst. The aqueous buffer was replaced by 10 to 20% by volume of DMSO. The results are shown in the following Table 14.

TABLE 14

| | | DMSO | | Conversion to |
|---|---|---|---|---|
| Ex. | Enzyme | (% by volume) | pH | vanillin (%) |
| 97 | peroxidase from Coprinus cinereus, batch 1 | 0 | 2 | 43 |
| 98 | peroxidase from Coprinus cinereus, batch 1 | 10 | 2 | 51 |
| 99 | peroxidase from Coprinus cinereus, batch 1 | 15 | 2 | 51 |
| 100 | peroxidase from Coprinus cinereus, batch 1 | 20 | 2 | 49 |
| 101 | horseradish peroxidase, batch 1 | 0 | 2 | 55 |
| 102 | horseradish peroxidase, batch 1 | 10 | 2 | 56 |
| 103 | horseradish peroxidase, batch 1 | 15 | 2 | 59 |
| 104 | horseradish peroxidase, batch 1 | 20 | 2 | 58 |

It has been shown that, with Coprinus cinereus peroxidase, DMSO resulted in a conversion increase of approximately 15-20% in all concentrations tested, while horseradish peroxidase hardly benefited from the presence of DMSO in this reaction (i.e. max. 5%).

### Examples 105 to 114

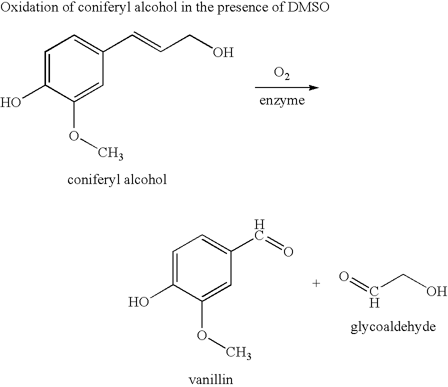

By analogy with Examples 97 to 104, coniferyl alcohol (4-hydroxy-3-methoxy cinnamyl alcohol) instead of isoeugenol was oxidized using the two enzymes, which provides an alternative synthetic route for obtaining vanillin by use of the inventive method and additionally provides glycolaldehyde (hydroxyacetaldehyde) as a side product, which is a useful reagent in protein chemistry. The aqueous buffer was again replaced, in this case by 5 to 20% DMSO. Table 15 shows the results obtained.

TABLE 15

| | | DMSO | | Conversion to |
|---|---|---|---|---|
| Ex. | Enzyme | (% by volume) | pH | vanillin (%) |
| 105 | peroxidase from Coprinus cinereus, batch 1 | 0 | 2 | 100 |
| 106 | peroxidase from Coprinus cinereus, batch 1 | 5 | 2 | 50 |
| 107 | peroxidase from Coprinus cinereus, batch 1 | 10 | 2 | 46 |
| 108 | peroxidase from Coprinus cinereus, batch 1 | 15 | 2 | 12 |
| 109 | peroxidase from Coprinus cinereus, batch 1 | 20 | 2 | 7 |
| 110 | horseradish peroxidase, batch 1 | 0 | 2 | 100 |
| 111 | horseradish peroxidase, batch 1 | 5 | 2 | 94 |
| 112 | horseradish peroxidase, batch 1 | 10 | 2 | 57 |
| 113 | horseradish peroxidase, batch 1 | 15 | 2 | 32 |
| 114 | horseradish peroxidase, batch 1 | 20 | 2 | 31 |

This clearly shows several facts. On the one hand, coniferyl alcohol was quantitatively oxidized to vanillin by use of either of the two enzymes as a catalyst, as long as no organic solvent was contained. On the other hand, while horseradish peroxidase still tolerated 5% DMSO well (6% decrease) and only showed clear activity losses afterwards (40 to 70%), Coprinus cinereus peroxidase decreased to 50% of its activity at only 5% DMSO. At 15% DMSO, approximately 90% of its activity were lost. These results are again gives evidence for the high specifity of the enzymatic catalysts in the method of the present invention.

Thus, the effectiveness of the enzymes in the inventive method has been clearly demonstrated. Based on the above teachings, the average artisan can easily determine the optimal conditions regarding pH, pressure, action of light, and organic solvent for individual, specific enzymes in specific oxidation reactions by means of optimization series. The invention thus constitutes an important contribution to the field of biocatalysis for the production of organic compounds.

### Materials and Supply Sources

trans-anethole: Sigma-Aldrich, Cat. 11, 787-0, Lot.: S16146-283
4-aminostyrene: Lancaster, 11845, 216-185-8, batch FA008716
2-bromstyrene: Sigma-Aldrich, 132683
2-methyl-1-phenyl-1-propene: Sigma-Aldrich, 282510, 13028 BE
indene: Merck-Schuchard, S17014 843, 8.20701.0005
isoeugenol: Sigma-Aldrich, 117206
conferyl alcohol: Sigma-Aldrich, 223735
peroxidase from Coprinus cinereus, batch 1: NovoNordisk A/S, Peroxidase SP 502, batch PPX 3829
peroxidase from Coprinus cinereus, batch 2: Novozymes, 51004, OON00008, (produced in Asp. oryzae)
peroxidase from Coprinus cinereus, batch 3: Biesterfeld Chemiehandel GmbH & Co. KG, “Baylase”
horseradish peroxidase, batch 1: Sigma-Aldrich, P2088
horseradish peroxidase, batch 2: Sigma-Aldrich, P8250, 031K74711
horseradish peroxidase, batch 3: Sigma-Aldrich, P6140, 051K7490
horseradish peroxidase, batch 4: Roche, POD10108090001, Lot.: 93350720
horseradish peroxidase, batch 5: Sigma-Aldrich, P8125, 031K7465
horseradish peroxidase, batch 6: Roche, POD10814407001, Lot.: 93396221
lignin peroxidase: Fluka, Lot. & Fillingcode 1239384, 32506 171, 42603
bovine milk peroxidase: Sigma-Aldrich, Lactoperoxidase from bovine milk, L-2005, Lot. 16H38311
chloroperoxidase from Caldaromyces fumago: Biochemika, 25810
bromoperoxidase from Corallina officinalis: Sigma-Aldrich, B2170, 123K3783
laccase from Rhus vernicifera: Sigma-Aldrich, L-2157, Lot.: 67H0281
laccase from Coriolus versicolor. Fluka, 38429, Lot. & Filling code 414571/1, 43001
laccase from Candida rugosa: Jülich Fine Chemicals Life Science Technology, Laccase CV, Ord. No. 14.10
laccase from Agaricus bisporus: Fluka, 40452, Lot. & Filling code 443928/1 42703431
DeniLite II Base laccase: Novozymes, OM30402613, Chemical Abstracts Service (CAS) Registry No.: 80498-15-3

### REFERENCES

[1] W. Yu, Y. Mei, Y. Kang, Z. Hua, Z. Jin, Org. Lett. 6, 3217-3219 (2004).
[2] B. R. Travis, R. S. Narayan, B. Borhan; J. Am. Chem. Soc. 124, 3824-3825 (2002).
[3] D. Yang, C. Zhang, J. Org. Chem. 66, 4814-4818 (2001).
[4] C.-M. Ho, W.-Y. Yu, C.-M. Che, Angew. Chem. 116, 3365-3369 (2004).
[5] R. S. Drago, B. B. Corden, C. W. Barnes, J. Am. Chem. Soc. 108, 2453-2454 (1986).
[6] G. Bourel, J.-M. Nicaud, B. Nthangeni, P. Santiago-Gomez, J.-M. Belin, F. Husson, Enzyme Microb. Technol. 35, 293-299 (2004).
[7] “Horseradish peroxidase in the presence of phenol as cosubstrate”, P. R. Ortiz de Montellano, L. A. Grab, Biochemistry 26, 5310-5314 (1987).
[8] “Engineered horseradish peroxidase”, S.-I. Ozaki, P. R. Ortiz de Montellano, J. Am. Chem. Soc. 117, 7056-7064 (1995).
[9] “Myeloperoxidase and Coprinus cinereus peroxidase with electron-deficient styrene derivatives”, A. Tuynman, J. L. Spelberg, I. M. Kooter, H. E. Schoemaker, R. Wever, J. Biol. Chem. 275, 3025-3030 (2000).
[10] “Chloroperoxidase from Caldariomyces fumago with tert-butyl hydroperoxide”, D. J. Bougioukou, I. Smonou, Tetrahedron Lett. 43, 339-342 (2002).
[11] D. J. Bougioukou, I. Smonou, Tetrahedron Lett. 43, 4511-4514 (2002).
[12] M. Takemoto, Y. Iwakiri, Y. Suzuki, K. Tanaka, Tetrahedron Lett. 45, 8061-8064 (2004).
[13] “Horseradish peroxidase with indoles”, K.-Q. Ling, L. M. Sayre, Bioorg. Med. Chem. 13, 3543-3551 (2005).
[14] “Employing β-carotene monooxygenase for epoxidation in a cascade with a hydrolase and further enzymes”, M. G. Leuenberger, C. Engeloch-Jarret, W.-D. Woggon, Angew. Chem. 113, 2684-2687 (2001).
[15] M. G. Leuenberger, C. Engeloch-Jarret, W.-D. Woggon, Angew. Chem. Int. Ed. 40, 2614-2617 (2001).
[16] T. D. H. Bugg, Tetrahedron 59, 7075-7101 (2003).
[17] M. Sono, M. P. Roach, E. D. Coulter, J. H. Dawson, Chem. Rev. 96, 2841-2888 (1996).
[18] “Quercetin 2,3-dioxygenase from Bacillus subtilis”, M. R. Schaab, B. M. Barney, W. A. Francisco, Biochemistry 45, 1009-1016 (2006).
[19] “Quercetin 2,3-dioxygenase from Aspergillus niger”, H.-K. Hund, J. Breuer, F. Lingens, J. Huttermann, R. Kappl, S. Fetzner, Eur. J. Biochem. 263, 871-878 (1999).
[20] “Indoleamine 2,3-dioxygenase and tryptophan 2,3-dioxygenase from mammals”, D. H. Munn, M. Zhou, J. T. Attwood, I. Bondarev, S. J. Conway, B. Marshall, C. Brown, A. L. Mellor, Science 281, 1191-1193 (1998).
[21] “A dioxygenase from Acinetobacter johnsonii is restricted to 1,3-diones”, G. D. Straganz, H. Hofer, W. Steiner, B. Nidetzky, J. Am. Chem. Soc. 126, 12202-12203 (2004).
[22] G. Straganz, A. Glieder, L. Brecker, D. W. Ribbons, W. Steiner, Biochem. J. 369, 573-581 (2003).
[23] “Lignostilbene-α,β-dioxygenase isozymes cleave various substituted stilbene derivatives”, S. Kamoda, T. Terada, Y. Saburi, Biosci. Biotechnol. Biochem. 62, 2575-2576 (1997).
[24] S. Kamoda, M. Samejima, Agric. Biol. Chem. 55, 1411-1412 (1991).
[25] S. Kamoda, Y. Saburi, Biosci. Biotechnol. Biochem. 57, 931-934 (1993).
[26] S. Kamoda, T. Terada, Y. Saburi, Biosci. Biotechnol. Biochem. 67, 1394-1396 (2003).
[27] S. Kamoda, T. Terada, Y. Saburi, Biosci. Biotechnol. Biochem. 69, 635-637 (2005).
[28] “Heme-dependent oxygenase cleaves double bonds of rubber (poly(cis-1,4-isoprene))”, R. Braaz, P. Fischer, D. Jendrossek, Appl. Environ. Microbiol. 70, 7388-7395 (2004).
[29] “β-Diketone cleavage”, G. Grogan, Biochem. J. 388, 721-730 (2005).
[30]H. Mang, J. Gross, M. Lara, C. Goessler, G. M. Gübitz, H. E. Schoemaker, W. Kroutil, Angew. Chem. Int. Ed. 45, 5201-5203 (2006).
[31]H. Mang, J. Gross, M. Lara, C. Goessler, G. M. Gübitz, H. E. Schoemaker, W. Kroutil, Angew. Chem. 118, 5325-5328 (2006).

1. A method for the oxidative cleavage of optionally substituted vinyl aromatic compounds of the following formula (1) 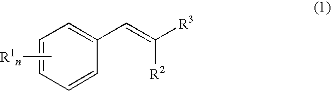
comprising oxidizing one or more compound(s) of formula (1) to aldehydes and ketones of the formulas (2) and (3), respectively, 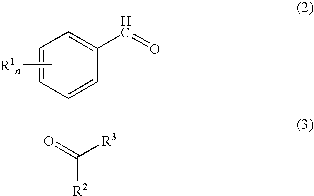
in the presence of molecular oxygen, and as catalysts, at least one enzyme selected from peroxidases and laccases, and at least one metalloprotein, according to the following general reaction scheme: 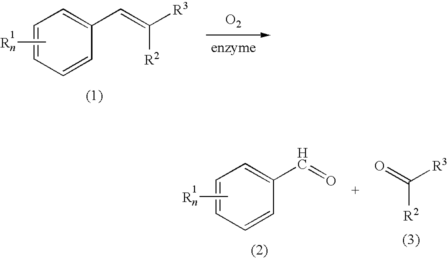
wherein n is an integer of 0 to 5, so that the aromatic ring in the compounds of formulas (1) and (2) may be substituted at the ortho, meta and/or para position(s) of the vinyl group with 0 to 5 substituents R¹ which may be identical or different and are selected from:
a) saturated or unsaturated hydrocarbon groups with 1 to 10 carbon atoms, wherein one or more carbon atoms are optionally substituted by a heteroatom selected from oxygen, nitrogen and sulfur, and which hydrocarbon groups are optionally further substituted with one or more substituents selected from the group consisting of C₁₋₆ alkyl groups, C₁₋₆ alkylene groups, C₁₋₆ alkoxy groups, amino, C₁₋₆ alkylamino groups, C₁₋₆ dialkylamino groups, halogens, hydroxy, oxo and cyano,
b) amino, C₁₋₆ alkylamino groups and C₁₋₆ dialkylamino groups, and
c) halogens, hydroxy and cyano,
wherein any two of the substituents R¹ may be linked to form an alicyclic or aromatic ring, and
wherein the substituents R² and R³ are each independently hydrogen or one of the options described in a), b) and c),
wherein R² and/or R³ in the formula (1) compound(s) may be linked to a substituent R¹ to form an alicyclic ring, in which case R² and R³ may each represent a chemical bond between the carbon atom of the vinyl group to which they are bound and the substituent R¹.

2. The method of claim 1, wherein the at least one enzyme is selected from fungal peroxidases and laccases, halogen peroxidases, lignin peroxidases, horseradish peroxidase, and bovine milk peroxidase.

3. The method of claim 2, wherein the at least one enzyme is selected from fungal peroxidases from Coprinus cinereus, from laccases from Coriolus versicolor, Agaricus bisporus and Candida rugosa, laccase from Rhus vernfera, chloroperoxidase from Caldariomyces fumago and bromoperoxidases from Streptomyces aureofaciens or Corallina officinalis.

4. The method of claim 2, wherein the at least one enzyme is selected from horseradish peroxidase, from peroxidases from Coprinus cinereus and laccases from Coriolus versicolor and Agaricus bisporus.

5. The method of claim 1, wherein the oxidation is carried out in a buffer.

6. The method of claim 5, wherein the buffer is selected from the group consisting of Bis-Tris buffer, acetate buffer, formate buffer, and phosphate buffer.

7. The method of claim 1, wherein the pH of the reaction is adjusted to 2 to 7.

8. The method of claim 1, wherein the oxidation is carried out at an O₂ overpressure.

9. The method of claim 8, wherein the oxidation is carried out at an O₂ overpressure of 1 to 6 bar.

10. The method of claim 1, wherein the oxidation is carried out under the action of light.

11. The method of claim 1, wherein the oxidation is carried out in the presence of an organic solvent or solvent mixture.

12. The method of claim 11, wherein the solvent or solvent mixture is used at a content of 1 to 20% by volume of the reaction mixture.

13. The method of claim 11 or 12, wherein the organic solvent is selected from the group consisting of C₁₋₄ alkanols, dimethyl sulfoxide, toluene, acetone, dioxane, tetrahydrofuran, dimethyl formamide, and mixtures thereof.

14. The method of claim 1, wherein vanillin is produced as the aldehyde of formula (2).

15. (canceled)

16. (canceled)

17. (canceled)
